(19) United States
(12) Patent Application Publication
(10) Pub. No.: US 2019/0298722 A1
(43) Pub. Date: Oct. 3, 2019

(54) USE OF AN INHIBITOR OF THE DE NOVO SYNTHESIS OF PURINES, IN THE TREATMENT OF ADENYLOSUCCINATE LYASE DEFICIENCY

(71) Applicants: ASSISTANCE PUBLIQUE-HOPITAUX DE PARIS, Paris (FR); INSTITUT NATIONAL DE LA SANTE ET DE LA RECHERCHE MEDICALE (INSERM), Paris (FR); UNIVERSITE PARIS DESCARTES, Paris (FR); IHU IMAGINE, Paris (FR)

(72) Inventors: Irène CEBALLOS-PICOT, Paris (FR); Laurence ROBEL, Paris (FR); Pascale DE LONLAY, Versailles (FR)

(21) Appl. No.: 16/324,454

(22) PCT Filed: Aug. 8, 2017
(86) PCT No.: PCT/FR2017/052213

§ 371 Date: Feb. 8, 2019

(30) Foreign Application Priority Data
Aug. 10, 2016 (FR) 1657690

(51) Int. Cl. A61K 31/519; A61K 9/00; A61P 25/00

(52) U.S. Cl. CPC A61K 31/519; A61P 25/00; A61K 9/0053

(57) ABSTRACT

The present invention relates to the use of an inhibitor of the de novo synthesis of purines (DNPS), in particular allopurinol, as a drug for the treatment of patients with adenylosuccinate lyase deficiency (ASLD).

The present invention relates to a novel therapeutic application of an inhibitor of de novo purine synthesis, and more particularly allopurinol, for the treatment of patients affected by adenylosuccinate lyase (ADSL) deficiency.

### CONTEXT OF THE INVENTION

Clinical Description of Adenylosuccinate Lyase Deficiency

Adenylosuccinate lyase (ADSL) deficiency (OMIM 103050) is a rare autosomal recessive disorder of purine metabolism, the main symptoms of which are learning disability, autistic disorders, and even encephalopathy with epileptic convulsive seizures. ADSL is a bifunctional enzyme involved in de novo purine synthesis, making it possible to catalyze the conversion of succinylaminoimidazole carboxamide ribotide (SAICAR) to AICAR and succinyl-AMP (S-AMP) to AMP (see FIG. 1).

ADSL deficiency leads to an accumulation of the succinylpurines succinylaminoimidazole carboxamide riboside (SAICAr) and succinyladenosine (S-Ado), that are dephosphorylated products of SAICAR and S-AMP respectively, in the body fluids.

Biochemically, diagnosis is thus carried out by means of detection of the 2 ADSL substrates called succinylpurines, SAICAr and S-Ado, in the body fluids. This detection is carried out more particularly in urine and CSF; the diagnosis is then confirmed by the sequencing of the ADSL gene.

Internationally, cases of more than 80 patients have been published but the incidence of the disorder remains unknown as this disorder is under-diagnosed. The probability of heterozygotes for a pathogenic ADSL mutation is approximately 1:10000 (Jurecka et al. 2015).

In France, 18 patients from 11 families have been diagnosed: 2 with the fatal neonatal form; 12 with the severe infantile form and 4 with the moderate form.

This disorder exhibits a wide spectrum of symptoms, with forms that progress slowly or very rapidly:

1—Neonatal Form:

The fatal neonatal form presents with neonatal encephalopathy, loss of spontaneous movements, respiratory distress, seizures that do not respond to any treatment, leading to death during the first weeks of life. Prenatal manifestations may exist with impaired intra-uterine growth, microcephaly, foetal hypokinesia, with the consequences thereof ranging from arthrogryposis to pulmonary hypoplasia.

2—ADSL Deficiency Type I: Severe Form

Patients with severe type I form present with a neurological picture characterized by severe psychomotor retardation, early epileptic seizures and autistic traits. Early seizures are the reason for initial consultation with a paediatric neurologist during the first months of life. Approximately half the patients with ADSL deficiency suffer from epilepsy that is difficult to treat. The most frequent autistic disorders are loss of eye contact, repetitive behaviours, agitation and self-injurious behaviour. Several stereotypies are described with regard to hand movements, repetitive manipulation of toys, grimacing, inappropriate laughter and sounds etc. Dysmorphic features are reported: microcephaly, intermittent divergent strabismus.

3—ADSL Deficiency Type II: Moderate Form

A slowly progressing form has been described (type II, moderate form) starting later, in the first months or years of life, with moderate psychomotor retardation and contact disorders that may be temporary. Progression can vary, with arrested development, loss of eye contact and in some patients, a vegetative state. Seizures, if present, occur later, often between the 2nd and 4th years of life, but seizures starting at the age of 9 years have recently been reported. Language difficulties involving minimal use of words contrast with significant means of non-verbal communication.

However, the symptoms of this disorder present on a continuum and despite the usefulness of classification in one of these 3 clinical categories, it is often difficult to place patients in a single one of these categories.

Diagnosis of ADSL Deficiency

The relatively wide clinical spectrum of ADSL deficiency reflects the difficulties in making a differential diagnosis from other neurological disorders that have convulsive seizures and encephalopathy in common.

For this reason, it is important to follow a protocol for a specific diagnosis of ADSL deficiency for children and new-borns presenting with:
hypotonia and acquired microcephaly,
unexplained psychomotor retardation,
unexplained developmental retardation, and/or
unexplained seizures that are particularly intractable

Diagnosis of ADSL deficiency requires:
demonstrating the presence of SAICAr and S-Ado in extracellular fluids (more particularly in urine since these metabolites are excreted) using the technique of high-performance liquid chromatography coupled with diode-array UV detection (HPLC-DAD) or high-performance liquid chromatography coupled with tandem mass spectrometry (HPLC-MS/MS),
followed by searching for mutations of the ADSL gene.

The HPLC-DAD and HPLC-MS/MS techniques allow simultaneous detection and quantification of SAICAr and S-Ado and should be the techniques of choice for a specific diagnosis of ADSL deficiency. The HPLC-DAD technique allows for accurate identification of components by their retention time and spectral analysis in comparison with the standard components available (Ceballos-Picot et al. 2015).

Etiopathogenesis

In humans, the gene coding for ADSL is on chromosome 22 (22q13.1q13.2), contains 13 exons and a typical promoter of housekeeping genes. In the majority of tissues, the ADSL gene is transcribed in two mRNAs produced by alternative splicing of exon 12.

The active ADSL protein is an enzyme composed of 484 amino acids. The variant originating from the alternative splicing with 59 missing amino acids (residues 397-456) is catalytically inactive and its biological role is not completely understood.

More than 50 different mutations of the ADSL gene have been described and their effects on the biogenesis of the ADSL protein, its stability and its activity have been characterized. Detailed and updated information on the mutations in identified patients can be found in a dedicated database accessible on the internet.

Current knowledge, essentially documented by means of the biochemical properties of mutant recombinant proteins associated with the fatal neonatal phenotype, make it possible to say that clinical severity is correlated with residual enzyme activity and stability of the mutant enzyme. Mutant ADSL proteins, in the fatal neonatal form, show the weakest residual activity in comparison with the severe (type I) or moderate (type II) forms, as well as thermal instability. Mutations mainly affect the amino acids of the active site or those that allow binding of the substrate. In all the cases studied to date, the combination of mutations produces an ADSL protein with persistent residual enzyme activity, a complete absence of ADSL enzyme activity probably being lethal.

Physiopathological Mechanisms

Hypotheses for explaining the pathogenesis are essentially based on the toxicity of a significant concentration of SAICAR in the brain and muscles and/or a deficiency in the production of nucleosides and nucleotides by means of de novo purine synthesis.

1—Toxic Effect of Succinylpurines

The major pathogenic effect is attributed to the toxicity of accumulating succinylpurines, more particularly SAICAR, which is shown to be toxic to the neurons.

It is also clearly shown that the ratio between the concentration of S-Ado and SAICAr in cerebrospinal fluid (S-Ado/SAICAr) is correlated with the clinical severity and with the following 3 phenotype groups:
in patients with the severe and fatal neonatal form, the S-Ado/SAICAr ratio is less than <1;
in patients with the severe infantile form presenting with epilepsy and autism, the S-Ado/SAICAr ratio is close to 1 (between 0.9 and 1.8);
in patients with the moderate form without epilepsy, the S-Ado/SAICAr ratio is greater than 2.

Observation of a less severe intellectual deficiency in patients with a similar concentration of SAICAr but with a higher S-Ado/SAICAr ratio suggests that SAICAr is the toxic compound and that S-Ado could have protective effects. This is confirmed by experimental data showing that the infusion of SAICAr into the brains of rats induces significant neuronal damage.

2—Nucleotide Deficiency Hypothesis

Deficiency of nucleotide synthesis caused by an ADSL deficiency, depending mainly on de novo purine synthesis, would be harmful to embryo development. However, a normal concentration of nucleotides has been measured in different tissues and cells of patients with ADSL deficiency; this hypothesis has therefore not been adopted. These results suggest that ADSL activity, although greatly reduced, does not limit the synthesis of nucleotides, and that the purine base salvage pathway involving the enzymes hypoxanthine-guanine phosphoribosyltransferase (HPRT), adenine phosphoribosyltransferase (APRT) and adenosine kinase (ADK) would compensate for the deficiency.

From all of these works, it is concluded that the increase in the concentration of the SAICAR metabolite, secondary to the ADSL deficiency, is implicated in the neurobehavioral disorders of ADSL deficiency due to its toxicity to the central nervous system.

Therapeutic Approaches

1—D-Ribose and Uridine

There are few articles on therapeutic trials. Trials of treatment with uridine or D-ribose, which would increase the production of phosphoribosylpyrophosphate (PRPP) and the synthesis of nucleotides, have been carried out on several patients and an improvement in motor coordination and seizure control in a 13-year-old child have been reported. However, these results have not been confirmed by other studies (Jurecka et al. 2015).

2—S-Adenosyl-1-Methionine

S-adenosyl-1-methionine (SAMe) has been assessed as a potential treatment for ADSL deficiency as a donor of adenosine, which is a nucleotide precursor. After 9 months of treatment with SAMe, there was no improvement in a patient's clinical and biological parameters.

3—Treatment of Epilepsy

The aim is to control and reduce the frequency of convulsive seizures and the intensity of seizures. The use of two or more anticonvulsants is often necessary in patients with ADSL deficiency. Resistance to these medicaments is common.

In conclusion, no trial for the treatment of ADSL deficiency has proved effective to date. There is therefore no specific and effective treatment for ADSL deficiency. Treatment options are currently limited to controlling convulsive seizures in the severe form.

### DESCRIPTION OF THE INVENTION

In this context, the inventors have put forward the hypothesis that inhibition of the first step of de novo purine synthesis (DNPS) could lead to a reduction in the quantity of succinylpurines produced, more particularly SAICAR, the toxicity of which to the neurons is established.

As this hypothesis was initially as uncertain as the (opposite) hypothesis that led to the unsuccessful clinical trials based on D-ribose and uridine, it was essential to test it using a molecule known to have no harmful side effects.

Allopurinol (1,2-dihydro-4H-pyrazolo[3,4-d]pyrimidin-4-one; CAS No: 315-30-0) is a medicament commonly used as a xanthine oxidase inhibitor (Zyloric; GlaxoSmithKline Laboratory). This medicament has been granted marketing authorization for the treatment of primary or secondary symptomatic hyperuricaemia, gout and uric lithiasis.

This molecule has another, little-known, biochemical property. In fact, allopurinol, a structural analogue of hypoxanthine, is also a substrate of hypoxanthine phosphoribosyltransferase (HPRT) and produces ribonucleotides of allopurinol halting SNDP by inhibiting the activity of PRPP amidotransferase, an enzyme of the first step of SNDP (Kelley and Wyngaarden, The Journal of Clinical Investigation, 1970) (FIG. 1).

Given the safety of this molecule, the inventors decided to test the following hypothesis: the negative feedback control of de novo purine synthesis by allopurinol could result in a reduction in the production of toxic succinylpurines in the brain, more particularly SAICAR, that accumulate in adenylosuccinate lyase (ADSL) deficiency. In this case, treating patients with ADSL deficiency with allopurinol would make it possible to reduce production of the toxic metabolite SAICAR.

Thus, 3 children who are full siblings were treated with allopurinol (200 mg/d) and after 6 and 12 months of treatment, the results showed a significant reduction of urinary SAICAr in the three children, accompanied by a substantial improvement in the behavioural disorders of the three children. The experimental data are detailed in the experimental part below.

These very positive results validate the initial hypothesis according to which inhibition of the first step of de novo purine synthesis 1—reduces the production of SAICAR and 2—has a favourable impact on the progress of patients affected by ADSL deficiency.

The present invention therefore firstly relates to the use of an inhibitor of de novo purine synthesis of (DNPS) as a medicament for treating patients affected by adenylosuccinate lyase (ADSL) deficiency.

In the context of the present invention, the terms “treat”, “treatment” etc. indicate an improvement in at least some of the symptoms of the disorder.

By way of non-limitative examples of inhibitors of DNPS, the natural substrates of the enzyme HPRT, i.e. hypoxanthine and guanine, as well as their structural analogues, may be mentioned. Other inhibitors of DNPS are capable of being used in the context of the present invention, such as, for example azaserine. This molecule, used in the treatment of certain cancers, directly inhibits de novo purine synthesis, like the competitive PRPP amidotransferase inhibitor and formylglycinamide ribonucleotide amidotransferase, thus upstream of SAICAR formation.

According to a preferred embodiment of the invention, the inhibitor of DNPS used to treat patients is a structural analogue of hypoxanthine such as, for example, allopurinol (1,2-dihydro-4H-pyrazolo[3,4-d]pyrimidin-4-one).

According to another preferred implementation of the invention, allopurinol is administered by the oral route. In an even more preferred manner, allopurinol is administered to the patient affected by ADSL deficiency in a dosage comprised between 100 and 400 mg/day for children and between 300 and 900 mg/d for adults. This dosage will be adapted depending on the weight (see tables 4 to 8 in the experimental part below) and the reduction in concentration of the toxic component targeted by the treatment: SAICAr.

As ADSL deficiency is a chronic disorder, in the context of the present invention treatment must be administered over a long period of time. According to a preferred implementation of the invention, allopurinol is administered daily for at least a year.

In order to limit the adverse effects for patients suffering from ADSL deficiency, and to promote their development, treatment with allopurinol is preferably started early. It can be started as soon as the diagnosis of ADSL deficiency is confirmed. Preferably, this treatment is started before 5 years of age. Even more preferably, treatment with allopurinol is started before the child affected by ADSL deficiency is 4 years old, 3 years old, 2 years old and ideally before the end of their first year. Of course, the practitioner will adapt the dosage in order to treat an infant.

The following examples illustrate the invention without however limiting the scope thereof.

### FIGURE LEGENDS

FIG. 1: Simplified diagram of purine metabolism and the de novo purine synthesis pathway. ADSL deficiency leads to an accumulation of SAICAr and S-Ado, that are dephosphorylated products of SAICAR and S-AMP respectively, in body fluids. Hypoxanthine phosphoribosyltransferase (HPRT), which accepts allopurinol as substrate (structural analogue of hypoxanthine) transforms it into a ribonucleotide of allopurinol. According to the inventors' hypothesis, the ribonucleotide of allopurinol has the ability to inhibit PRPP amidotransferase, an enzyme of the first step of de novo purine synthesis, thus leading to a decrease in the production of SAICAr and S-Ado. ADSL: Adenylosuccinate Lyase; APRT: adenine phosphoribosyltransferase; ADK: adenosine kinase; HPRT: hypoxanthine phosphoribosyltransferase; XO: xanthine oxidase. The symbol (-) indicates inhibition.

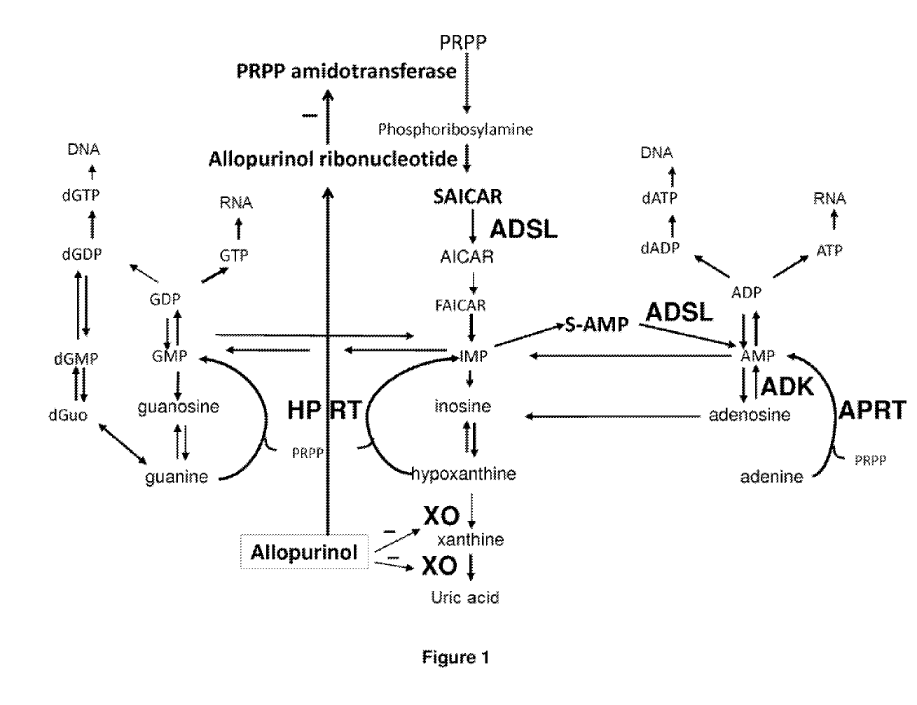

### EXAMPLES

### Example 1: Administration of Allopurinol for One Year to Three Children Who are Full Siblings Affected by ADSL Deficiency

Three children who are full siblings were treated with allopurinol (200 mg/d) and, after 6 months, the results show a significant reduction of urinary SAICAr (Table 1) in the three children. This reduction is accompanied by a substantial improvement in the behavioural disorders assessed using the Conners and Vineland scales (Table 3). A reduction in hyperactivity has also been reported by the parents and teachers, as well as an overall improvement in attention (Table 3).

1.1 Materials and Methods

Assay of SAICAr and S-Ado Metabolites

SAICAr and S-Ado metabolites in urine were assayed using HPLC-DAD and HPLC coupled with tandem mass spectrometry (HPLC-MS/MS) according to the techniques recently described (Ceballos-Picot et al. 2015; Zikanova et al. 2014).

Clinical Assessment

Clinical characterization of the patients was carried out before starting treatment (T1) in accordance with the ICD 10 classification, using standardized tools:
A semi-structured interview by means of the Autism Diagnostic Interview (ADI-R): this is a semi-structured interview intended to classify symptoms reported by parents in the domain of communication, social interaction, restricted and stereotyped behaviours or sensory issues. Scores are established for the current period and, if necessary, for the period when the difficulties were most serious, between the ages of 4 and 5 years. A score is established for each of the three domains based on responses provided by the parents and the judgement of the interviewer. Scores above the thresholds in the 3 domains support a diagnosis of autism, and in one or two domains support a diagnosis of a pervasive developmental disorder. Scores below the thresholds in the 3 domains make it possible to rule out diagnosis of an autism spectrum disorder if the ADI-R results converge with the clinical assessment of the child.
A semi-structured interview using the Vineland 2 scale: this is a semi-structured interview which makes it possible to assess the adaptive skills of the child in the domains of communication, daily life, social skills and motor skills. From the description of their child's daily living skills, equivalent developmental ages and standardized scores are calculated, making it possible to describe the developmental and adaptive profile of the child.
A developmental assessment using the Psychoeducational Profile (PEP 3): this is a direct assessment of the skills of the child in several domains of their development, particularly suited to children who display a developmental disability. Six domains of development are assessed: verbal and preverbal cognition (VPC), expressive language (EL), receptive language (RL), gross motor skills (GM), fine motor skills (FM) and visual-motor imitation (VMI). The results are expressed in terms of equivalent developmental age and emerging score, when the exercises are partially completed. The number of emerging skills, or the difference between the actual ages and the emerging scores, are evidence of the development potential of the children.
The Conners scale: this is an assessment scale intended to quantify the symptoms of inattentiveness, agitation, impulsiveness, anxiety, somatization, behavioural difficulties and learning difficulties. There are several versions of the questionnaire, one for parents, the other for teachers or professionals. The scoring makes it possible to obtain an overall standardized score, considered to be significant when it is greater than 1.5.

Clinical re-assessments were carried out 6 months and one year after treatment with allopurinol and compared to the first assessment in the absence of treatment with allopurinol. They comprised:
At 6 months: scoring of the Vineland and Conners scales
At 12 months: scoring of the Vineland and Conners scales and completion of PEP-3.

Inclusion of Patients

Three children who are full siblings, with parents who are unrelated, of high socio-economic status, were included in the study. All three are carriers of ADSL deficiency: a girl aged 9 years 7 months (patient 1; P1) and two boys aged 6 years 2 months (patient 2; P2) and 4 years 8 months (patient 3; P3) respectively.

The clinical characteristics of these patients are described in point 1.3 below.

Treatment with Allopurinol

The patients received allopurinol daily by the oral route (200 mg/d).

1.2 Assessment of the Biochemical Effectiveness of Allopurinol by Assay of SAICAr and S-Ado in Urine

The inventors have developed a technique for assaying SAICAr and S-Ado by means of HPLC-MS/MS (high-performance liquid chromatography coupled with tandem mass spectrometry) that is very specific and sensitive based on a publication by a Czech team (Zikanova et al. 2014). Another HPLC technique with UV detection (HPLC-DAD) was also used (comparison of methods) and similar results were obtained, showing a significant reduction in SAICAr and S-Ado after 6 and 12 months of treatment with allopurinol in these 3 patients with ADSL deficiency.

TABLE 1
Urinary assay of SAICAr and S-Adenosine by means of
high-performance liquid chromatography coupled with
tandem mass spectrometry (HPLC-MS/MS) before treatment
(T1) and after 6 months (T2) and 12 months (T3) of
treatment with allopurinol in 3 patients with ADSL
deficiency who are full siblings (P1; P2; P3).

| | S-Adenosine | SAICAr | % | % | |
|---|---|---|---|---|---|
| | (μmol/mmol | (μmol/mmol | variation | variation | S-Ado/ |
| Patient | creat) | creat) | S-Adenosine | SAICAr | SAICAr |
| P1 T1 | 55.4 | 37.7 | | | 1.47 |
| P1 T2 | 51.6 | 32.9 | −6.9 | −12.7 | 1.57 |
| P1 T3 | 36.4 | 19.4 | −34.3 | −48.6 | 1.88 |
| P2 T1 | 116.3 | 95.4 | | | 1.22 |
| P2 T2 | 79.3 | 57.9 | −31.8 | −39.3 | 1.37 |
| P2 T3 | 63.7 | 46.8 | −45.2 | −51.0 | 1.36 |
| P3 T1 | 148.6 | 167.9 | | | 0.89 |
| P3 T2 | 113.3 | 96.3 | −23.8 | −42.7 | 1.18 |
| P3 T3 | 98.2 | 72.9 | −33.9 | −56.6 | 1.35 |

1.3 Clinical Description of Patients Before Treatment with Allopurinol

The three patients were included in the study with the consent of their parents. The diagnosis of adenylosuccinate lyase (ADSL) deficiency was given for the three children after the birth of the 3rd child, based on biochemical testing (detection of succinyl purines in urine by the Bratton-Marshall test, then confirmed by specific measurement of the succinylpurines SAICAr and S-Ado in urine by HPLC-DAD) and was confirmed by detection of two distinct mutations of the ADSL gene, the Y114H mutation of paternal origin, and the G418A mutation of maternal origin.

Patient 1 (P1), aged 9 years 7 months at the time of inclusion, was born by caesarean section for breech presentation. The first clinical signs associated delayed psychomotor development with the acquisition of walking at 22 months, absence of language and the appearance of motor stereotypies at the age of 10 months. A first assessment carried out at the age of 18 months concluded a global developmental delay, without epilepsy. The ophthalmologic assessment also highlighted ametropia of the hypermetropia type with amblyopia without strabismus. The neurological, genetic and metabolic etiological assessment carried out was non-contributory. At the age of 7, the diagnosis of ADSL deficiency was finally given based on the combination of developmental disorders in the 3 children, based on a positive Bratton-Marshall test in the other two children and a genetic analysis of the ADSL gene revealing the Y114H mutation of paternal origin, and the G418A mutation of maternal origin.

Patient 2 (P2), aged 6 years 2 months at the time of inclusion, was also born by caesarean section. He presented with delayed psychomotor development with acquisition of walking at 18 months, associated with delayed acquisition of oral language predominantly affecting expression and psychomotor agitation without symptoms of autism or epilepsy. He also presented with hypermetropia that was corrected.

Patient 3 (P3), aged 4 years 8 months at the time of inclusion, was born by planned caesarean section. He was a calm baby, who also presented with delayed psychomotor development with delayed acquisition of walking after 18 months and delayed language predominantly affecting expression. He presented with a malaise at the age of 21 months, which revealed a subdural haematoma, that progressed favourably. He did not have symptoms of autism, in particular no difficulties with social interactions or restricted or invasive interests, nor epilepsy.

Assessment of the patients included the use of several standardized tools, intended to accurately describe the symptomatology of these children: the presence of autism spectrum disorder (ADI-R), their level of development (PEP3 scale) their level of functioning in daily life (Vineland Scale), symptoms of inattentiveness, agitation, impulsiveness, anxiety, somatization and learning difficulties (Conners Scale).

The clinical characteristics of the patients are described in Table 2.

TABLE 2
Clinical characteristics of patients at inclusion.

| Patient | P1 | P2 | P3 |
|---|---|---|---|
| Diagnosis | F84.8, F72.1 | F71.1, F80.2, F90 | F71.1, F80.2, F90 |
| sex | F | M | M |
| age | 9 years 7 months | 6 years 2 months | 4 years 8 months |
| ADI-R | | | |
| Social interactions (>10) | 8 | 6 | 4 |
| Communication (>7) | 7 | 5 | 2 |
| Restricted and stereotyped | 8 | 0 | 0 |
| interests (>3) | | | |
| Vineland | | | |
| Communication | 67 | 57 | 65 |
| Daily life | 69 | 81 | 75 |
| Social skills | 73 | 79 | 76 |
| Motor skills | — | 61 | 67 |
| TOTAL | 69 | 69 | 71 |
| PEP-3 | | | |
| VPC | 2 years 6 months | 2 years 3 months | 1 year 6 months |
| EL | 1 year 7 | 1 year 3 months | 1 year 4 months |
| RL | 1 year 7 | 1 year 8 months | 1 year 5 months |
| FM | 2 years 2 | 1 year 9 months | 1 year 4 months |
| GM | 2 years 5 | 2 years 3 months | 2 years |
| VMI | 2 years 8 | 6 years | 2 years 1 month |
| DA | | 2 years 7 months | 2 years |
| CONNERS - PARENTS | | | |
| Behavioural difficulties | 6 | 1 | 2 |
| Learning difficulties | 14 | 7 | 9 |
| Somatization | 0 | 0 | 0 |
| Impulsiveness, hyperactivity | 8 | 4 | 9 |
| Anxiety | 1 | 2 | 2 |
| v-score | 29 | 14 | 22 |
| CONNERS - TEACHERS | | | |
| Behavioural difficulties | 2 | 16 | 14 |
| Impulsiveness, hyperactivity | 10 | 14 | 11 |
| Inattentiveness, passivity | 10 | 11 | 12 |

ADI-R: Autism Diagnostic Interview-Revised.
Vineland: Standardized scores obtained on the Vineland scale (average of 100).
PEP-3: Psychoeducational Profile 3.
VPC: verbal and preverbal

cognition; EL: expressive language; RL: receptive language; FM: fine motor skills; GM: gross motor skills; VMI: visual-motor imitation; DA: overall developmental age. CONNERS: Conners Scale completed by teachers and parents.

For patient 1 (P1), the ADI-R scores are significant in the domain of communication and restricted and stereotyped interests, placing her in the category of pervasive developmental disorders not otherwise specified (PDD-NOS) or autism spectrum disorder (ASD). The Vineland scale scores place her in the mild deficiency range on the functional level, the greatest deficiency being in the domain of communication. Her development level on the PEP3 scale places her in a range of moderate deficiency. At the time of inclusion, she had a diagnosis of pervasive developmental disorder not otherwise specified (F84.8), associated with a moderate intellectual disability (F71.1) according to the International Classification of Diseases (ICD 10). She also had symptoms of agitation and inattentiveness, which do not fall within the definition of hyperkinetic disorder due to exclusion criteria.

For patient 2 (P2) the ADI-R scores are not significant in any of the 3 domains, making it possible to exclude an autism spectrum disorder. The Vineland scale scores place him in the mild deficiency range as regards function, the greatest deficiency being in the domain of communication. His development level on the PEP3 scale is heterogeneous, quantifying good visual-motor imitation contrasting with significant deficiencies in the levels of expressive and receptive language, and marginally better fine and gross motor skills. The overall development quotient (DA/CAx100), places him in a range of moderate deficiency, with a development quotient (DQ) of 42. As regards behaviour, the hyperactivity index is significant for teachers, but not for the parents.

For patient 3 (P3) the ADI-R scores are not significant in any of the 3 domains, making it possible to exclude an autism spectrum disorder. The Vineland scale scores place him in the mild deficiency range as regards function, the greatest deficiency being in the domain of communication. His development level on the PEP3 scale is fairly homogenous, but relatively weaker for expressive and receptive language and fine motor skills. The overall development quotient (DA/CAx100), places him in a range of moderate deficiency, with a development quotient (DQ) of 43. As regards behaviour, the hyperactivity index is significant for teachers and for the parents.

Patients 2 and 3 have a diagnosis of mild intellectual disability (F70.1) associated with an oral language acquisition disorder of the receptive type (F80.2) and a hyperkinetic disorder (F90), according to the diagnostic criteria of ICD 10.

1.4 Clinical Progress of the Patients Undergoing Treatment with Allopurinol

Clinical progress at 200 mg of allopurinol was measured for each child based on the Conner and Vineland scale scores before treatment (T1), at 6 months (T2) and at 12 months of treatment (T3) with regard to behaviour and function.

Developmental progress was measured by means of developmental ages on the PEP3 scale before treatment (T1) and after a year of treatment (T3).

Progress in the scores reflects clinical progress, with a reduction in hyperactivity scores reported by the parents, and an overall improvement in scores on the Vineland and PEP3 scales.

TABLE 3
Progress of scores on the Conners, Vineland and PEP3 scales
after 6 months (T2) and 12 months (T3) of treatment with
allopurinol compared with scores before treatment (T1).

| | Patient | | | | | | | | |
|---|---|---|---|---|---|---|---|---|---|
| | Patient 1 | | | Patient 2 | | | Patient 3 | | |
| Time | T1 | T2 | T3 | T1 | T2 | T3 | T1 | T2 | T3 |
| Chronological age | 9.7 | 10.1 | 10.7 | 6.2 | 6.8 | 7.2 | 4.8 | 5.2 | 5.8 |
| Conners | | | | | | | | | |
| Teachers | 2.2 | 1.5 | 1 | 2 | 1.5 | 1.4 | 1.9 | 1.8 | 1 |
| Parents | 2.3 | 1.6 | 1.8 | 1 | 0.8 | 0.5 | 1.7 | 1.2 | 1 |
| Vineland | | | | | | | | | |
| Communication | 67 | 65 | 75 | 57 | 72 | 75 | 65 | 69 | 74 |
| Daily life | 69 | 71 | 74 | 81 | 87 | 87 | 75 | 87 | 87 |
| Social skills | 73 | 66 | 75 | 79 | 83 | 83 | 76 | 83 | 88 |
| Motor skills | — | — | — | 61 | 67 | 85 | 67 | 70 | 72 |
| TOTAL | 69 | 76 | 74 | 69 | 79 | 82 | 71 | 77 | 80 |
| PEP3 | | | | | | | | | |
| VPC | 2.6 | — | 3 | 2.3 | — | 2.3 | 1.6 | — | 2.4 |
| EL | 1.7 | — | 1.8 | 1.3 | — | 1.7 | 1.4 | — | 2.3 |
| RL | 1.7 | — | 2.3 | 1.8 | — | 2.7 | 1.5 | — | 2 |
| GM | 2.2 | — | 2.8 | 1.9 | — | 3.3 | 1.4 | — | 3.4 |
| FM | 2.5 | — | 2.8 | 2.3 | — | 4.0 | 2 | — | 3.6 |
| VMI | 2.8 | — | 3.3 | 6 | — | 6.0 | 2.1 | — | 3.3 |

During treatment, the following were observed:
A reduction in scores on the Conners scale for the 3 patients, on the scales completed by the parents and on the scales completed by teachers. Teachers were not informed of the treatment. The scores relating to impulsiveness and hyperactivity, learning difficulties and behavioural disorders were those that progressed most on the parents' scale; the scores for impulsiveness and hyperactivity and that for inattentiveness progressed most on the scales completed by teachers.
An improvement in the standardized scores on the Vineland scale in all domains for the 3 patients, more pronounced for communication.
A progression in the equivalent developmental ages, although the rate of progression remains slow with respect to chronological age.

Progress of Adaptive Behaviours

The progress of adaptive levels measured using the Vineland scale scores show:
An improvement in the 3 patients in the domain of “listening and understanding” communication. For patients P2 and P3, the improvement took place in the first 6 months of treatment, between T1 and T2, whereas it took place later in patient P1. The domains of “speaking” and “reading and writing” showed little improvement.
An improvement in the 3 patients in the domain of “personal care” in daily life and little or no improvement in the “living in the community” component. The domain of “taking care of the home” tended to improve for the older patient, this item having little relevance for the younger children.
An improvement in the 3 patients in the domain of social skills for the domains of “contact with others” and “free play”, while the “coping” dimension did not improve.

Progress of Developmental Levels

The progress of developmental levels measured on the PEP3 scale shows:
A clear improvement in the domain of fine motor skills, limited improvement in the domains of expressive language, verbal and preverbal cognition and gross motor skills and a stagnation in the domain of expressive language and visual-motor imitation in patient P1, the eldest patient.
An improvement of more than a year in developmental age in the domain of gross motor skills, approximately a year for fine motor skills and receptive language, and little or no improvement for expressive language, verbal and preverbal cognition and visual-motor imitation for patient P2.
An improvement in all of the domains of development for patient P3, the youngest patient, particularly in the domain of expressive language and preverbal and verbal cognition, with a gain of more than a year in developmental level.

Progress of Behaviours:

Assessment of the behavioural disorders, learning difficulties, symptoms of impulsiveness and hyperactivity, inattentiveness and passivity measured by the scores obtained on the Conners scales, completed independently at 3 points in time T1, T2 and T3 by the parents and teachers shows:
An overall improvement for the 3 patients, measured by a reduction in the v-score for the 3 patients on the scale completed by the parents. This improvement reflects in particular a reduction in the impulsiveness and attention difficulties, and an improvement in learning, while the symptoms of somatization, anxiety and behavioural difficulties remain mild.
A reduction in hyperactivity and impulsiveness in the 3 patients, on the scale completed by teachers.
A slight reduction in passivity and attentional difficulties in patient P1.
A very clear improvement in behavioural difficulties, a reduction in passivity and attentional difficulties in patients P2 and P3.

1.5 Summary of the Predictable and Known Benefits and Risks for Persons Taking Part in the Research

In summary, during treatment with allopurinol, the inventors observed a clinical improvement in the 3 patients, more significant in the youngest patient (P3). This improvement reflects in particular a reduction in attentional difficulties and hyperactivity, measured by the parents and teachers, an improvement in adaptive functioning in particular with regard to communication, and a relative improvement in developmental level, relating in particular to comprehension of language and fine and gross motor skills.

This protocol is only beneficial, given the convincing results in the 3 patients already treated and the absence of a treatment for this pathology to date.

The risks are limited to the formation of xanthine crystals and therefore kidney stones if the daily intake of liquids is insufficient. It will therefore be necessary to monitor the crystalluria at 1, 3, 6 and 12 months of treatment then every year if treatment with allopurinol continues.

### Example 2: Continuation of Study

Given the success of treatment with allopurinol in the 3 children affected by ADSL deficiency described in Example 1, a hospital clinical research protocol (HCRP) is put in place in order to assess the effectiveness of treatment with allopurinol in 12 other patients presenting with an ADSL deficiency in France.

Administration will be by the oral route, in the theoretical dose of 10 mg/Kg/day in one or more doses, without exceeding 400 mg/day in children and 900 mg/day in adults over 12 months (cf. Tables 4 to 8). It will be started progressively in order to avoid the risk of allergies: 100 mg/day for one month, i.e. 1 tablet to be administered in the morning, then progressive increase (intermediate dose prescribed for M2) as a function of the target dose to be achieved at M3. Treatment will be continued in the absence of undesirable effects from M3 to M6 then from M6 to M12.

If there are cutaneous marks, no adaptation of the dosage is provided for; the treatment will be stopped immediately.

The dosage can be increased at M6 up to a theoretical dose of 20 mg/Kg/day without exceeding 400 mg/day in children and 900 mg/day in adults if the urinary assay for SAICAr and S-Ado metabolites at M3 does not show a reduction of at least 70%. Otherwise, the dosage will remain that prescribed at M3, i.e. a theoretical dose of 10 mg/Kg/day.

TABLE 4
Chart of doses to be administered to an adult with normal kidney function for a dosage
of 10 mg/kg/d: ceiling dose at 900 mg/d. From M 0 to M 3, starting at 100 mg/d at
M 1, then progressive increase to M 2 then full dose at M 3, then from M 6 to M 12
if the urinary assay for SAICAR and S-Ado shows a reduction of at least 70%.

| | Interval of | | | | | | Number of 100 |
|---|---|---|---|---|---|---|---|
| | actual dose | Dose to be | Number | Dose to be | Number | Dose to be | mg tablets per |
| | administered | administered | of 100 mg | administered | of 100 mg | administered | day at M 3 (and |
| Weight of | at M 3 | (mg/d) | tablets per | (mg/d) | tablets per | (mg/d) | at M 6 and M 12 |
| patient (kg) | (mg/Kg/d) | at M 1 | day at M 1 | at M 2 | day at M 2 | at M 3 | if applicable) |
| 40 to 44.5 | 9 to 10 | 100 | 1 | 200 | 2 | 400 | 4 |
| 44.6 to 54.5 | 9 to 11 | 100 | 1 | 300 | 3 | 500 | 5 |
| 54.6 to 64.5 | 9 to 11 | 100 | 1 | 300 | 3 | 600 | 6 |
| 64.6 to 74.5 | 9 to 11 | 100 | 1 | 400 | 4 | 700 | 7 |
| 74.6 to 84.5 | 9 to 11 | 100 | 1 | 400 | 4 | 800 | 8 |
| from 84.6 kg | 9 to 11 | 100 | 1 | 400 | 4 | 900 | 9 |
| and above | | | | | | | |

TABLE 5
Scale of doses to be administered to a child with normal kidney function for a dosage
of 10 mg/kg/d: ceiling dose at 400 mg/d. From M 0 to M 3, starting at 100 mg/d at M
1, then progressive increase to M 2 then full dose at M 3, then from M 6 to M 12 if
the urinary assay for SAICAR and S-Ado in urine shows a reduction of at least 70%.

| | Interval of | | | | | | Number of 100 |
|---|---|---|---|---|---|---|---|
| | actual dose | Dose to be | Number | Dose to be | Number | Dose to be | mg tablets per |
| | administered | administered | of 100 mg | administered | of 100 mg | administered | day at M 3 (and |
| Weight of | at M 3 | (mg/d) | tablets per | (mg/d) | tablets per | (mg/d) | at M 6 and M 12 |
| patient (kg) | (mg/kg/D) | at M 1 | day at M 1 | at M 2 | day at M 2 | at M 3 | if applicable) |
| 7 to 14.5 | 7 to 14 | 100 | 1 | 100 | 1 | 100 | 1 |
| 14.6 to 24.5 | 8 to 14 | 100 | 1 | 200 | 2 | 200 | 2 |
| 24.6 to 34.5 | 9 to 12 | 100 | 1 | 200 | 2 | 300 | 3 |
| from 34.6 kg | 5 to 12 | 100 | 1 | 200 | 2 | 400 | 4 |
| and above | | | | | | | |

TABLE 6
Chart of doses to be administered to an adult with normal kidney
function for a dosage of 20 mg/kg/d: ceiling dose at 900 mg/d.
From M 3 to M 6, then from M 6 to M 12 if the urinary assay
for SAICAR and S-Ado shows a reduction of at least 70%.

| | | | Number of 100 mg |
|---|---|---|---|
| | Interval of | | tablets per day |
| | actual dose | Dose to be | from M 3 to M 6 |
| Weight of | administered | administered | (then from M 6 to |
| patient (kg) | (mg/kg/D) | (mg/d) | M 12 if applicable) |
| 40 to 42.2 | 19 to 20 | 800 | 8 |
| from 42.3 kg | 9 to 21 | 900 | 9 |
| and above | | | |

TABLE 7
Chart of doses to be administered to a child with normal kidney
function for a dosage of 20 mg/kg/d: ceiling dose at 400 mg/d.
From M 3 to M 6, then from M 6 to M 12 if the urinary assay
for SAICAR and S-Ado shows a reduction of at least 70%.

| | | | Number of 100 mg |
|---|---|---|---|
| | Interval of | | tablets per day |
| | actual dose | | from M 3 to M 6 |
| Weight of | administered | Dose | (then from M 6 to |
| patient (kg) | (mg/kg/d) | (mg/d) | M 12 if applicable) |
| 7 to 12.2 | 16 to 29 | 200 | 2 |
| 12.3 to 17.2 | 17 to 24 | 300 | 3 |
| from 17.3 kg | 5 to 23 | 400 | 4 |
| and above | | | |

TABLE 8
Chart indicating the number of tablets taken and distribution
over time as a function of the prescribed dose.

| | | Distribution of tablets |
|---|---|---|
| Dose | Number of 100 mg | taken throughout |
| administered | tablets to be | the day (morning/ |
| (mg/d) | administered per day | midday/evening) |
| 100 | 1 | 1 0 0 |
| 200 | 2 | 1 1 0 |
| 300 | 3 | 1 1 1 |
| 400 | 4 | 1 1 2 |
| 500 | 5 | 1 2 2 |
| 600 | 6 | 2 2 2 |
| 700 | 7 | 2 2 3 |
| 800 | 8 | 2 3 3 |
| 900 | 9 | 3 3 3 |

In the presence of renal insufficiency, the adapted dosage will be that recommended by the marketing authorization for allopurinol.

The dosage must be adapted as a function of creatinine clearance:

TABLE 9
Maximum dose recommended for patients with renal insufficiency.

| Creatinine clearance | Maximum recommended dose |
|---|---|
| 80 < Cl Cr < 100 ml/mn | 300 mg/d |
| 40 < Cl Cr < 80 ml/mn | 200 mg/d |
| 20 < Cl Cr < 40 ml/mn | 100 mg/d |
| Cl Cr < 20 ml/mn | 100 mg/1 day in 2 |

At the end of 12 months of treatment, it will (or will not) be continued. The choice will be left to the discretion of the lead researcher.

Treatment with allopurinol will be given without associated administration of adenine.

1. A method for treatment of a patient affected by adenylosuccinate lyase (ADSL) deficiency, comprising administering an inhibitor of de novo purine synthesis (DNPS) to a patient in need thereof.

2. The method according to claim 1, wherein the inhibitor of DNPS is a substrate of hypoxanthine phosphoribosyltransferase (HPRT).

3. The method according to claim 1, wherein the inhibitor of DNPS is a structural analogue of hypoxanthine.

4. The method according to claim 1, wherein the inhibitor of DNPS is allopurinol.

5. The method according to claim 4, wherein allopurinol is administered orally.

6. The method according to claim 4, wherein allopurinol is administered to a child, in a dosage comprised between 100 and 400 mg/day.

7. The method according to claim 4, wherein allopurinol is administered in a dosage of 200 mg/day.

8. The method according to claim 4, wherein allopurinol is administered to a patient aged under 5 years.

9. The method according to claim 4, wherein allopurinol is administered to an adult, in a dosage comprised between 300 and 900 mg/day.

10. The method according to claim 4, wherein allopurinol is administered daily for at least one year.
